(19) United States
(12) Patent Application Publication
(10) Pub. No.: US 2010/0069532 A1
(43) Pub. Date: Mar. 18, 2010

(54) PROCESS FOR PREPARING POLYMERIZABLE CARBOXYLIC ESTERS WITH ALKOXY GROUPS

(75) Inventors: Paola Uribe Arocha, Mannheim (DE); Joachim Pakusch, Speyer (DE); Stefan Becker, Mannheim (DE); Thomas Goetz, Leimersheim (DE); Sylke Haremza, Neckargemuend (DE); Rolf Gulden, Frankenthal (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/516,628

(22) PCT Filed: Dec. 3, 2007
(86) PCT No.: PCT/EP07/63127

§ 371 Date: May 28, 2009

(30) Foreign Application Priority Data
Dec. 8, 2006 (EP) 06125742.4

(51) Int. Cl. C08K 3/00; C07C 69/54; C07C 69/60; C08F 20/26; C08F 22/20

(52) U.S. Cl. 524/5; 560/224; 560/198; 526/320

(57) ABSTRACT

A process for preparing free-radically polymerizable carboxylic esters by reacting ethyllenically unsaturated carboxylic acids, carboxylic anhydrides or carbonyl halides (referred to collectively as carboxylic acid component) with a hydroxyl compound composed of at least 60% by weight of C₂ to C₄ alkoxy groups (and referred to for short as polyalkoxy compound), which comprises said reacting taking place
in the presence of a polymerization inhibitor and
of a reducing agent.

The present invention relates to a process for preparing free-radically polymerizable carboxylic esters by reacting ethylenically unsaturated carboxylic acids, carboxylic anhydrides or carbonyl halides (referred to collectively as carboxylic acid component) with a hydroxyl compound composed of at least 60% by weight of O₂ to C₄ alkoxy groups (and referred to for short as polyalkoxy compound), which comprises said reacting taking place
in the presence of a polymerization inhibitor and
of a reducing agent.

The invention additionally relates to copolymers which comprise the carboxylic esters and to the use of the copolymers as a plasticizing additive in cementitious preparations.

Free-radically polymerizable carboxylic esters, particularly monoesters of poly-C₂-C₄ alkylene glycols with acrylic acid or methacrylic acid, also referred to below as poly-C₂-C₄ alkylene glycol mono(meth)acrylic esters, are used for example in the preparation of comb polymers having poly-C₂-C₄ alkylene ether side chains. The latter polymers have surface-active properties which predestine them for diverse utilities: for example, as laundry detergent additives such as incrustation inhibitors, graying inhibitors, and soil release agents, and also as paint ingredients and as formulating additives for active-ingredient preparations in medicine and in crop protection.

Anionic comb polymers having poly-C₂-C₄ alkylene ether side chains and carboxylate groups on the polymer backbone, especially those with C₁-C₁₀ alkylpolyethylene glycol side chains, find use, for example, as plasticizers for mineral-based binding building materials, especially for cementitious binding building materials such as mortar, cement-bound renders and, in particular, concrete.

Poly-C₂-C₄ alkylene glycol mono(meth)acrylic esters are typically prepared by esterifying an OH-bearing poly-C₂-C₄ alkylene glycol with acrylic acid or methacrylic acid.

In the literature there are descriptions of different processes.

Part of the description of DE-A 1110866 concerns the reaction of monoalkylpolyalkylene glycols with chlorides of ethylenically unsaturated carboxylic acids, the acid chloride being used in excess. The crude ester product obtained, as will be appreciated, comprises as yet unreacted excess acid chloride, which disrupts further reactions and must be removed by means of a costly and inconvenient distillation. The quality of the poly-C₂-C₄ alkylene glycol mono(meth)acrylic esters prepared in this way is not satisfactory.

U.S. Pat. No. 4,075,411 describes the preparation of alkylphenoxy(polyethylene glycol) monoesters of olefinically unsaturated carboxylic acids by esterification of polyethylene glycol mono(alkylphenyl)ethers with the corresponding acid in the presence of p-toluenesulfonic acid or by reaction with the acid chloride in the presence of an amine. The conversions attained and the quality of the alkylphenoxy(polyethylene glycol) monoesters prepared in this way are not satisfactory.

WO 01/74736 describes a process for preparing copolymers of poly-C₂-C₄ alkylene glycol mono(meth)acrylic esters, with acrylic acid or methacrylic acid, by copolymerizing these monomers, the poly C₂-C₄ alkylene glycol mono(meth)acrylic esters being prepared by reacting polyalkylene glycols with (meth)acrylic anhydrides in the presence of amines. For this reaction the anhydride is used in an excess of at least 10 mol %, based on the stoichiometry of the reaction. In spite of this excess, the rate of the esterification is low. In their own investigations, moreover, the inventors have shown that the esterification conversions attained are low and that the esters prepared in this way comprise not only free anhydride but also considerable amounts of unreacted polyalkylene glycols, which adversely affect the quality of the polymers subsequently prepared, particularly with regard to their use as concrete plasticizers.

WO 2006/024538 describes a process which involves reacting acrylic anhydride and/or methacrylic anhydride with a poly-C₂-C₄ alkylene glycol compound, bearing at least one OH group, in the presence of a base, the base being selected from basic compounds having a solubility in of not more than 10 g/l at 90° C., and using (meth)acrylic anhydride A and poly-C₂-C₄ alkylene glycol compound P in an A:P molar ratio in the range from 1:1 to 1.095:1. This process enabled the quality of the carboxylic esters and the conversion rate as well to be improved.

WO 2006/024538 also describes the accompanying use of a polymerization inhibitor during the esterification. Suitable polymerization inhibitors often require oxygen for their activity; furthermore, oxygen itself may also act as an inhibitor. A disadvantage when oxygen is present, however, is the formation of peroxides. In polyalkylene oxides, peroxides cause ether cleavage, for example, and as a result of unwanted crosslinking reactions they lead to carboxylic esters having more than one polymerizable group. Polyfunctional carboxylic esters of this kind, in subsequent polymerization, lead to instances of crosslinking and result in a broad molar weight distribution.

For many applications, not least for use as plasticizing additives in cementitious preparations, uniform copolymers are advantageous.

It is an object of the present invention, therefore, to provide a process for preparing free-radically polymerizable carboxylic esters which on copolymerization produce uniform copolymers and which are suitable particularly as a plasticizing additive in cementitious preparations.

The process defined at the outset was found accordingly.

### The Constituents of the Carboxylic Ester

Suitability as carboxylic acid component is possessed by all free-radically polymerizable carboxylic acids, carboxylic anhydrides or carbonyl halides. These may be, for example, dicarboxylic acids or their anhydrides, for example maleic acid, maleic anhydride, fumaric acid, itaconic acid or itaconic anhydride. They are preferably monocarboxylic acids, such as acrylic acid or methacrylic acid, more preferably dimeric anhydrides of the monocarboxylic acids, and especially acrylic anhydride or methacrylic anhydride.

The polyalkoxy compound has preferably one or two, more preferably two, hydroxyl groups which react esterifyingly with the carboxylic acid components.

The polyalkoxy compound is composed preferably of at least 80% by weight of C₂ to C₄ alkoxy groups. Preferred C₂-C₄ alkoxy groups are ethoxy groups, propoxy groups or mixtures thereof, more preferably ethoxy groups. In one preferred embodiment at least 70%, more preferably at least 90%, and in particular 100% by weight of the alkoxy groups are ethoxy groups.

The polyalkoxy compound has in general at least 3, frequently at least 5, and in particular at least 10 and in general not more than 400, frequently not more than 300, e.g., 10 to 200, and in particular 10 to 150 alkoxy groups. The compounds may be linear or branched and have in general on average at least one, typically terminal, free OH group in the molecule. The remaining end groups may for example be OH groups, alkyloxy groups having preferably 1 to 10 C atoms, phenyloxy or benzyloxy groups, acyloxy groups having preferably 1 to 10 C atoms, O—SO₃H groups or O—PO₃H₂ groups, of which the latter two groups may also take the form of anionic groups. In one preferred embodiment a polyalkyloxy compound is employed in which one end group is an OH group and the other or further end group or groups is or are (an) alkyloxy group(s) having 1 to 10 and in particular having 1 to 4 C atoms such as ethoxy, n-propoxy, isopropoxy, n-butoxy, 2-butoxy or tert-butoxy, and especially methoxy.

Preference is given to linear polyalkoxy compounds having approximately one free OH group per molecule (i.e., about 0.9 to 1.1 free OH groups on average). Compounds of this kind can be described by the general formula P:

HO-(A-O)ₙ—R¹ (P)

in which n indicates the number of repeating units and is generally a number in the range from 3 to 400, in particular in the range from 5 to 300, more preferably in the range from 10 to 200, and very preferably in the range from 10 to 150,
A is C₂-C₄ alkylene such as 1,2-ethanediyl, 1,3-propanediyl, 1,2-propanediyl, 1,2-butanediyl or 1,4-butanediyl, and
R¹ is hydrogen, alkyl having preferably 1 to 10 and in particular 1 to 4 C atoms, phenyl, benzyl, acyl (═C(O)-alkyl) having preferably 1 to 10 C atoms, SO₃H groups or PO₃H₂, especially C₁-C₁₀ alkyl and more preferably C₁-C₄ alkyl and especially methyl or ethyl.

With particular preference A is CH₂—CH₂ or

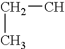

With very particular preference A is CH₂—CH₂

An especially preferred embodiment of the invention, accordingly, concerns a process in which the alkoxy compound is a polyethylene glycol mono(C₁-C₁₀ alkyl)ether, in other words a mono-C₁-C₁₀ alkyl ether, in particular a mono-C₁-C₄ alkyl ether, and especially the methyl or ethyl ether, of a linear polyethylene glycol.

The polyalkoxy compound preferably has a number-average molecular weight (determined by means of GPC) in the range from 250 to 20 000 and in particular in the range from 400 to 10 000.

The free-radically polymerizable carboxylic ester is, accordingly, preferably the acrylic or methacrylic ester of the above polyalkoxy compound.

The preparation process of the carboxylic ester

In accordance with the invention the polymerizable carboxylic ester is prepared in the presence of a polymerization inhibitor.

Preferred polymerization inhibitors are those selected from sterically hindered nitroxides, cerium(III) compounds, and sterically hindered phenols and their mixtures, and also mixtures thereof with oxygen.

Suitable more particularly are, in particular, phenols such as hydroquinone, hydroquinone monomethyl ether, especially sterically hindered phenols such as 2,6-di-tert-butylphenol or 2,6-di-tert-butyl-4-methylphenol, and also thiazines such as phenothiazine or methylene blue, cerium(III) salts such as cerium(III) acetate, and nitroxides, especially sterically hindered nitroxides, i.e., nitroxides of secondary amines which bear 3 alkyl groups on each of the C atoms adjacent to the nitroxide group, with 2 of these alkyl groups, particularly those not located on the same C atom, forming a saturated 5- or 6-membered ring with the nitrogen atom of the nitroxide group and/or the carbon atom to which they are attached, such as, for example, in 2,2,6,6-tetramethylpiperidine-1-oxyl (TEMPO) or 4-hydroxy-2,2,6,6-tetramethylpiperidine-1-oxyl (OH-TEMPO), mixtures of the aforementioned inhibitors, mixtures of the aforementioned inhibitors with oxygen, in the form for example of air, and mixtures of mixtures of the aforementioned inhibitors with oxygen, in the form for example of air. Preferred inhibitors are the afore-mentioned sterically hindered nitroxides, cerium(III) compounds, and sterically hindered phenols and their mixtures with one another, and also mixtures of such inhibitors with oxygen, and mixtures of mixtures of these inhibitors with oxygen, in the form for example of air. Particular preference is given to inhibitor systems which comprise at least one sterically hindered nitroxide and a further component selected from a sterically hindered phenol and a cerium(III) compound, and also mixtures thereof with oxygen, in the form for example of air.

The amount of the polymerization inhibitor may in particular be up to 2% by weight, based on the total amount of carboxylic acid component and alkoxy compound. The inhibitors are used advantageously in amounts of 10 ppm to 1000 ppm, based on the total amount of carboxylic acid component and polyalkoxy compound. In the case of inhibitor mixtures, these figures are based on the total amount of the components, with the exception of oxygen.

In accordance with the invention the polymerizable carboxylic ester is also prepared in the presence of a reducing agent.

Suitable reducing agents include, in particular, phosphorus or sulfur compounds.

Sulfur compounds include for example sodium disulfide, sodium thiosulfate or mercaptans, such as butyl mercaptan, mercaptoacetic acid, mercaptopropionic acid or mercaptoethanol.

The reducing agent comprises with particular preference phosphorus compounds, by which are meant both organic and inorganic phosphorus compounds. The inorganic phosphorus compounds for use in accordance with the invention preferably comprise the oxo acids of phosphorus and their salts which are dispersible or soluble in the reaction medium, preferably their alkali metal, alkaline earth metal or ammonium salts.

Examples of suitable inorganic phosphorus compounds are as follows:

phosphinic acid (H₂PO₂) and the salts derived therefrom, such as sodium phosphinate (monohydrate), potassium phosphinate, ammonium phosphinate; hypodiphosphonic acid (H₄P₂O₄) and the salts derived therefrom; phosphonic acid (H₃PO₃) and the salts derived therefrom such as sodium hydrogen phosphonate, sodium phosphonate, potassium hydrogen phosphonate, ammonium hydrogen phosphonate, ammonium phosphonate; diphosphonic acid (H₄P₂O₅) and the diphosphonates derived therefrom; hypodiphosphoric acid (H₄P₂O₆) and the hypodiphosphates derived therefrom; diphosphoric acid (H₄P₂O₇) and the diphosphates derived therefrom, and also polyphosphoric acids and their salts, such as sodium triphosphate. The carboxylic esters are preferably prepared in the presence of phosphinic acid (H₃PO₂) or the salts derived therefrom, examples being sodium hydrogen phosphonate, sodium phosphonate, potassium hydrogen phosphonate, potassium phosphonate, ammonium hydrogen phosphonate, and ammonium phosphonate. Particular preference is given to sodium phosphinate monohydrate and/or phosphonic acid. Phosphorus compounds further comprise organophosphorus compounds as well, such as urea phosphate, methanediphosphonic acid, propane-1,2,3-triphosphonic acid, butane-1,2,3,4-tetraphosphonic acid, polyvinylphosphonic acid, 1-aminoethane-1,1-diphosphonic acid, diethyl (1-hydroxyethyl)phosphonate, diethyl hydroxymethylphosphonate, 1-amino-1-phenyl-1,1-diphosphonic acid, aminotrismethylenetriphosphonic acid, ethylenediaminotetramethylenetetraphosphonic acid, ethylenetriaminopentamethylenepentaphosphonic acid, ethylenediaminotetramethylenetetraphosphonic acid, ethylenetriaminopentamethylenepentaphosphonic acid, ethylenediaminotetramethylenetetraphosphonic acid, ethylenetriaminopentamethylenepentaphosphonic acid,

1-hydroxyethane-1,1-diphosphonic acid, phosphonoacetic and phosphonopropionic acids and their salts, diethyl phosphite, dibutyl phosphite, diphenyl phosphite, triethyl phosphite, tributyl phosphite, triphenyl phosphite, and tributyl phosphate. Also suitable are ethylenically unsaturated phosphorus compounds such as vinyl phosphonate, methyl vinylphosphonate, ethyl vinylphosphonate, vinyl phosphate, allyl phosphonate or allyl phosphate.

Preferred organophosphorus compounds are 1-hydroxyethane-1,1-diphosphonic acid and its disodium and tetrasodium salts, aminotrismethylenetriphosphonic acid, and also the pentasodium salt, and ethylenediaminotetramethylenetetraphosphonic acid and its salt.

Often it is advantageous to combine two or more phosphorus compounds, such as, for example, sodium phosphinate monohydrate with phosphonic acid, phosphonic acid with disodium 1-hydroxyethane-1,1-diphosphonate and/or aminotrimethylenetriphosphonic acid and/or 1-hydroxyethane-1,1-diphosphonic acid. They can be mixed with one another in any desired proportion and used in the polymerization.

The amount of reducing agent, preferably of phosphorus compound, is preferably 0.01 to 5 parts by weight, preferably 0.03 to 3 parts by weight, in particular 0.05 to 2 parts by weight per 100 parts by weight of carboxylic acid component and polyalkoxy compound.

The preparation of the polymerizable carboxylic ester preferably takes place, furthermore, at a reduced oxygen content.

The reaction takes place preferably in the presence of a gas mixture having an oxygen concentration of 1% to 15% by volume.

The reaction of the anhydride with the compound P can be carried out in all apparatus typical for such reactions, such as in a stirred tank, in stirred tank cascades, autoclaves, tube reactors or compounders, for example. The reaction space available in the apparatus is preferably not filled completely with the reaction mixture; in general, only a maximum of 90% by volume, in particular only a maximum of 80% by volume, is filled with the reaction mixture. The remaining space is occupied by the gas mixture. The gas mixture is preferably passed continuously through the reaction space.

Otherwise the preparation takes place preferably in accordance with the process described in WO 2006/024538.

Consequently the polymerizable carboxylic ester is preferably prepared in the presence of a base.

The base is preferably selected from basic compounds which have a solubility in the polyalkoxy compound of not more than 10 g/l, more preferably not more than 5 g/l, at 90° C.

The examples of inventively suitable bases include hydroxides, oxides, carbonates, and hydrogen carbonates of monovalent or divalent metal cations, particularly of elements from main groups I and II of the periodic table, i.e., of Li⁺, Na⁺, K⁺, Rb⁺, Cs⁺, Be²⁺, Mg²⁺, Ca²⁺, Sr²⁺, and Ba²⁺, and also of monovalent or divalent transition metal cations such as Ag⁺, Fe²⁺, Co²⁺, Ni²⁺, Cu²⁺, Zn²⁺, Cd²⁺, Sn²⁺, Pb²⁺, and Ce²⁺. Preference is given to the hydroxides, oxides, carbonates, and hydrogen carbonates of cations of the alkali and alkaline earth metals and also of Zn²⁺, and in particular of Mg²⁺ or Ca²⁺, and with particular preference of Na⁺ or K⁺. Preferred among these are the hydroxides and carbonates of these metal ions, particularly the alkali metal carbonates and alkali metal hydroxides, and especially sodium carbonate, potassium carbonate, potassium hydroxide, and sodium hydroxide. Also suitable in particular is lithium hydroxide and lithium carbonate. The base is used preferably in an amount of 0.05 to 0.5 base equivalents and in particular in an amount of 0.1 to 0.4 base equivalents, based on the polyalkoxy compound, although larger quantities of base, up to 1 base equivalent for example, are generally no disadvantage. It should be borne in mind here that in the case of hydroxides and hydrogen carbonates the base equivalents correspond to the molar equivalents employed, whereas 1 mol equivalent of a carbonate or oxide corresponds in each case to 2 base equivalents.

For preparing the free-radically polymerizable carboxylic ester it is preferred to add the carboxylic acid component in excess. The molar ratio of the reactive carboxylic acid groups of the carboxylic acid components to the hydroxyl groups of the polyalkyloxy compound can be for example 1:0.5 to 5:1, preferably 1:1 to 5:1, and very preferably 1.2:1 to 4:1. The excess carboxylic acid components are copolymerized in the subsequent copolymerization. It should be borne in mind that (meth)acrylic anhydride is a dimer having two carboxylic acid groups per (meth)acrylic anhydride. The (meth)acrylic anhydride expression refers, here and below, not only to acrylic anhydride or methacrylic anhydride but also to mixtures thereof. (Meth)acrylic anhydride is used preferably in excess relative to the polyalkylene oxide compound (corresponding to a much larger excess relative to the reactive carboxylic acid groups). The excess of (meth)acrylic anhydride will in one preferred embodiment not exceed 9.5 mol %, preferably 9 mol %, in particular 8.5 mol %, and especially 8 mol %, based on 1 mol of compound P (polyalkylene oxide); in other words, the amount of (meth)acrylic anhydride is at most 1.095 mol, preferably not more than 1.09 mol, in particular not more than 1.085 mol, and especially not more than 1.08 mol per mole of compound P. It is preferred to use at least 1.005 mol, in particular at least 1.01 mol, and with particular preference at least 1.02 mol of (meth)acrylic anhydride per mole of compound P.

The reaction of the carboxylic acid components with the polyalkoxy compound takes place preferably at temperatures in the range of 0 and 150° C., in particular in the range from 20 to 130° C., and more preferably in the range of 50 and 100° C. The pressure prevailing during the reaction is of minor importance to the success of the reaction, and is situated in general in the range from 800 mbar to 2 bar and frequently at ambient pressure. It is preferred to carry out the reaction in an inert gas atmosphere.

The reaction of the carboxylic acid components with the polyalkoxy compound is carried out preferably until the conversion of the compound P employed is at least 80%, in particular at least 90%, and more preferably at least 95%. The reaction times required to achieve such a conversion will generally not exceed 5 h and are frequently less than 4 h. The conversion can be monitored by ¹H NMR spectroscopy of the reaction mixture, preferably in the presence of a strong acid such as trifluoroacetic acid.

The reaction of the carboxylic acid components with the polyalkoxy compound can be carried out in bulk, i.e., without the addition of solvents, or in inert solvents or diluents. Inert solvents are generally aprotic compounds. The inert solvents include unhalogenated or halogenated aromatic hydrocarbons such as toluene, o-xylene, p-xylene, cumene, chlorobenzene, ethylbenzene, technical mixtures of alkylaromatics, and aliphatic and cycloaliphatic hydrocarbons such as hexane, heptane, octane, isooctane, cyclohexane, cycloheptane, technical aliphatics mixtures, and also ketones such as acetone, methyl ethyl ketone, cyclohexanone, and also ethers such as tetrahydrofuran, dioxane, diethyl ether, tert-butyl methyl ether, and mixtures of the aforementioned solvents, such as toluene/hexane, for example. It is preferred to operate without solvent or with only very small amounts of solvent, generally of less than 10% by weight, based on the ingredients; in other words, in bulk.

The reaction mixture therefore preferably comprises less than 5% by weight of solvents such as water or organic solvents.

It has proven advantageous to carry out the reaction of the carboxylic acid components with the polyalkoxy compound in a reaction medium that comprises less than 0.2% by weight and in particular less than 1000 ppm of water (determined by Karl-Fischer titration). The term “reaction medium” refers to the mixture of the reactants A and P with the base and also with any solvent and inhibitor employed. In the case of ingredient materials which contain moisture it has been found appropriate to remove the water prior to the reaction, by means for example of distillation and with particular preference by distillation with addition of an organic solvent that forms a low-boiling azeotrope with water. Examples of solvents of this kind are the aforementioned aromatic solvents such as toluene, o-xylene, p-xylene, cumene, benzene, chlorobenzene, ethylbenzene, and technical aromatics mixtures, and also aliphatic and cycloaliphatic solvents such as hexane, heptane, and cyclohexane, and also technical aliphatics mixtures and mixtures of the aforementioned solvents.

For the reaction a typical procedure is to react the reaction mixture comprising the polyalkoxy compound and the carboxylic acid component and the base and, if appropriate, solvent, inhibitor, and reducing agent in a suitable reaction vessel at the temperatures indicated above. It is preferred to introduce the polyalkoxy compound and the base and also, if appropriate, the solvent as an initial charge and to add the carboxylic acid component to it.

If the ingredients comprise water, the water will preferably be removed prior to the addition of the carboxylic acid components.

The reaction of the polyalkoxy compound with the carboxylic acid components leads to a mixture which comprises the polymerizable carboxylic ester and if appropriate, depending on the amount of carboxylic acid components employed, comprises polymerizable carboxylic acid components as well.

The Copolymers and their Use

The free-radically polymerizable carboxylic ester obtained is used preferably for preparing homopolymers or copolymers.

In particular it is possible to use the free-radically polymerizable carboxylic esters without prior isolation from the esterification product mixture.

In the case of the copolymers it is possible simply to add the other monomers required to the product mixture.

Preferred copolymers are synthesized from:

10% to 99.9%, more preferably 50% to 99%, and very preferably 70% to 97% by weight of the free-radically polymerizable carboxylic ester (A),

0.1% to 50%, more preferably 1% to 30%, and very preferably 2% to 15% by weight of acrylic acid or methacrylic acid (B), and

0% to 30%, more preferably 0% to 20%, and very preferably 0% to 10% by weight of further monomers (C)

Examples of monomers C) are:
C1 monoethylenically unsaturated monocarboxylic and dicarboxylic acids having 3 to 8 C atoms such as crotonic acid, isocrotonic acid, maleic acid, fumaric acid, and itaconic acid,
C2 alkyl esters of monoethylenically unsaturated mono- and di-C₃-C₈-carboxylic acids, particularly of acrylic acid and of methacrylic acid, with C1-C10 alkanols or C₃-C₁₀ cycloalkanols such as methyl acrylate, ethyl acrylate, n-propyl acrylate, isopropyl acrylate, n-butyl acrylate, isobutyl acrylate, tert-butyl acrylate, n-hexyl acrylate, 2-ethylhexyl acrylate, cyclohexyl acrylate, and the corresponding methacrylic esters,
C3 hydroxyalkyl esters of monoethylenically unsaturated mono- and di-C₃-C₈-carboxylic acids, particularly of acrylic acid and methacrylic acid, such as 2-hydroxyethyl acrylate, 3-hydroxypropyl acrylate, 4-hydroxybutyl acrylate, 2-hydroxyethyl methacrylate, 3-hydroxypropyl methacrylate, and 4-hydroxybutyl methacrylate,
C4 monoethylenically unsaturated nitriles such as acrylonitrile,
C5 vinylaromatic monomers such as styrene and vinyltoluenes,
C6 monoethylenically unsaturated sulfonic acids and phosphonic acids and salts thereof, especially their alkali metal salts such as vinylsulfonic acid, allylsulfonic acid, methallylsulfonic acid, styrenesulfonic acid, 2-acryloyloxyethanesulfonic acid, 2-acrylamido-2-methylpropanesulfonic acid, vinylphosphonic acid, allylphosphonic acid, 2-acryloxyethanephosphonic acid, and 2-acrylamido-2-methylpropanephosphonic acid, and also
C7 amino-bearing monomers and their protonation products and their quaternization products, such as 2-(N,N-dimethylamino)ethyl acrylate, 2-(N,N-dimethylamino)ethyl methacrylate, 3-(N,N-dimethylamino)propyl acrylate, 2-(N,N-dimethylamino)propyl methacrylate, 2-(N,N,N-trimethylammonio)ethyl acrylate, 2-(N,N,N-trimethylammonio)ethyl methacrylate, 3-(N,N,N-trimethylammonio)-propyl acrylate, and 2-(N,N,N-trimethylammonio)propyl methacrylate, in the form of their chlorides, sulfates, and methosulfates.

Preferred monomers C are the monomers C1, C3, and C6. The fraction of monoethyllenically unsaturated monomers as a proportion of the total amount of monomers to be polymerized will generally not exceed 30% by weight and in particular not exceed 10% by weight. In one particularly preferred embodiment zero or less than 1% by weight, based on the total amount of the monomers C to be polymerized, is employed, based on the total amount of the monomers to be polymerized.

Furthermore, in order to increase the molecular weight of the polymers it can be useful to carry out the copolymerization in the presence of small amounts of polyethylenically unsaturated monomers having for example 2, 3 or 4 polymerizable double bonds (crosslinkers). Examples thereof are diesters and triesters of ethylenically unsaturated carboxylic acids, particularly the bis- and trisacrylates of diols or polyols having 3 or more OH groups, examples being the bisacrylates and the bismethacrylates of ethylene glycol, diethylene glycol, triethylene glycol, neopentyl glycol or polyethylene glycols. Crosslinkers of this kind are used if desired in an amount of in general 0.01% to 5% by weight, based on the total amount of the monomers to be polymerized. It is preferred to use less than 0.01% by weight and in particular no crosslinker monomers.

The copolymerization of the carboxylic ester with acrylic acid and/or methacrylic acid and, if appropriate, further monomers takes place typically in the presence of compounds which form free radicals and which are referred to as initiators. Compounds of this kind are used typically in amounts up to 30%, preferably 0.05% to 15%, and in particular 0.2% to 8% by weight, based on the monomers to be polymerized. In the case of initiators composed of two or more constituents (initiator systems, as in the case for example of redox initiator systems) the weight figures above relate to the sum of the components.

Examples of suitable initiators include organic peroxides and hydroperoxides, additionally peroxodisulfates, percarbonates, peroxide esters, hydrogen peroxide, and azo compounds. Examples of initiators are hydrogen peroxide, dicyclohexyl peroxydicarbonate, diacetyl peroxide, di-tert-butyl peroxide, diamyl peroxide, dioctanoyl peroxide, didecanoyl peroxide, dilauroyl peroxide, dibenzoyl peroxide, bis(o-tolyl) peroxide, succinyl peroxide, methyl ethyl ketone peroxide, di-tert-butyl hydroperoxide, acetylacetone peroxide, butyl peracetate, tert-butyl permaleate, tert-butyl perisobutyrate, tert-butyl perpivalate, tert-butyl peroctoate, tert-butyl perneodecanoate, tert-butyl perbenzoate, tert-butyl hydroperoxide, cumene hydroperoxide, tert-butyl perneodecanoate, tert-amyl perpivalate, tert-butyl perpivalate, tert-butylperbenzoate, tert-butyl peroxy-2-ethylhexanoate, and diisopropylperoxydicarbamate; additionally lithium, sodium, potassium, and ammonium peroxodisulfates, azo initiators 2,2′-azobis-isobutyronitrile, 2,2′-azobis(2-methylbutyronitrile), 2,2′-azobis[2-methyl-N-(2-hydroxyethyl)propionamide], 1,1′-azobis(1-cyclohexanecarbonitrile), 2,2′-azobis(2,4-dimethylvaleronitrile), 2,2′-azobis(N,N′-dimethyleneisobutyroamidine) dihydrochloride, and 2,2′-azobis(2-amidinopropane) dihydrochloride, and also the redox initiator systems elucidated hereinbelow.

Redox initiator systems comprise at least one peroxide compound in combination with a redox coinitiator, such as a sulfur compound having a reducing action, examples being bisulfites, sulfites, thiosulfates, dithionites and tetrathionates of alkali metals or of ammonium compounds. Thus it is possible to use combinations of peroxodisulfates with alkali metal hydrogen sulfites or ammonium hydrogen sulfites, an example of such a combination being ammonium peroxodisulfate and ammonium disulfite. The amount of the peroxide compound relative to the redox coinitiator is 30:1 to 0.05:1.

The initiators can be employed alone or in a mixture with one another, examples being mixtures of hydrogen peroxide and sodium peroxodisulfate.

The initiators may either be soluble in water or else insoluble or sparingly soluble in water. For polymerization in an aqueous medium it is preferred to use water-soluble initiators, i.e. initiators which in the concentration typically employed for the polymerization are soluble in the aqueous polymerization medium. Such initiators include peroxodisulfates, azo initiators with ionic groups, organic hydroperoxides having up to 6 C atoms, acetone hydroperoxide, methyl ethyl ketone hydroperoxide and hydrogen peroxide, and also the aforementioned redox initiators.

In combination with the initiators and/or with the redox initiator systems it is additionally possible to use transition metal catalysts, such as salts of iron, cobalt, nickel, copper, vanadium, and manganese. Examples of suitable salts include iron(II) sulfate, cobalt(II) chloride, nickel(II) sulfate, or copper(I) chloride. Relative to the monomers, the reductive transition metal salt is used in a concentration of 0.1 ppm to 1000 ppm. Thus it is possible to use combinations of hydrogen peroxide with iron(II) salts, such as, for example, 0.5% to 30% of hydrogen peroxide and 0.1 to 500 ppm of Mohr's salt.

In the case of copolymerization in organic solvents as well it is possible, in combination with the abovementioned initiators, to use redox coinitiators and/or transition metal catalysts in addition, examples being benzoin, dimethylaniline, ascorbic acid, and organic-solvent-soluble complexes of heavy metals such as copper, cobalt, iron, manganese, nickel, and chromium. The amounts typically used of redox coinitiators and/or transition metal catalysts are approximately 0.1 to 1000 ppm, based on the amounts of monomers employed.

In order to place a check on the average molecular weight of the polymers obtainable in accordance with the invention it is often useful to carry out the copolymerization of the invention in the presence of regulators. For this purpose it is possible to use typical regulators, particularly organic compounds comprising SH groups, especially water-soluble compounds comprising SH groups, such as 2-mercaptoethanol, 2-mercaptopropanol, 3-mercaptopropionic acid, cysteine, N-acetylcysteine, and also phosphorus(III) or phosphorus(I) compounds such as alkali metal hypophosphites or alkaline earth metal hypophosphites, sodium hypophosphite for example, and also hydrogen sulfites such as sodium hydrogen sulfite. The polymerization regulators are used in general in amounts of 0.05% to 10% by weight, in particular 0.1% to 2% by weight, based on the monomers. Preferred regulators are the aforementioned SH-bearing compounds, especially water-soluble SH-bearing compounds such as 2-mercaptoethanol, 2-mercaptopropanol, 3-mercaptopropionic acid, cysteine and N-acetylcysteine. With these compounds it has proven particularly appropriate to use them in an amount of 0.05% to 2% by weight, in particular 0.1% to 1% by weight, based on the monomers. The aforementioned phosphorus(III) and phosphorus(I) compounds and also the hydrogen sulfites will be used typically in larger amounts, 0.5% to 10% by weight for example and 1% to 8% by weight in particular, based on the monomers to be polymerized. Through the choice of appropriate solvent it is also possible to influence the average molecular weight. For instance, polymerization in the presence of diluents having benzylic or allylic H atoms leads to a reduction in the average molecular weight, as a result of chain transfer.

The copolymerization may take place according to the customary polymerization processes, including solution polymerization, precipitation polymerization, suspension polymerization or bulk polymerization. Preference is given to the method of solution polymerization, i.e. polymerization in solvents or diluents.

The suitable solvents or diluents include not only aprotic solvents, examples being the aforementioned aromatics such as toluene, o-xylene, p-xylene, cumene, chlorobenzene, ethylbenzene, technical mixtures of alkylaromatics, aliphatics and cycloaliphatics such as cyclohexane and technical aliphatics mixtures, ketones such as acetone, cyclohexanone, and methyl ethyl ketone, ethers such as tetrahydrofuran, dioxane, diethyl ether, and tert-butyl methyl ether, and C₁-C₄ alkyl esters of aliphatic C₁-C₄ carboxylic acids such as methyl acetate and ethyl acetate, but also protic solvents such as glycols and glycol derivatives, polyalkylene glycols and their derivatives, C₁-C₄ alkanols, examples being n-propanol, n-butanol, isopropanol, ethanol or methanol, and also water and mixtures of water with C₁-C₄ alkanols such as, for example, isopropanol/water mixtures. The copolymerization process takes place preferably in water or in a mixture of water with up to 60% by weight of

C₁-C₄ alkanols or glycols as solvents or diluents. With particular preference water is used as the sole solvent.

The copolymerization process is carried out preferably in the substantial or complete absence of oxygen, preferably in a stream of inert gas, as for example in a nitrogen stream.

The copolymerization process can be carried out in the apparatus typical for polymerization methods. Such apparatus includes stirred tanks, stirred tank cascades, autoclaves, tube reactors, and compounders.

The copolymerization process takes place typically at temperatures in the range from 0 to 300° C., preferably in the range from 40 to 120° C. The duration of polymerization is typically in the range from 0.5 h to 15 h and in particular in the range from 2 to 6 h. The pressure prevailing during the polymerization is of minor importance to the outcome of the polymerization and is situated generally in the range from 800 mbar to 2 bar and frequently at ambient pressure. When using volatile solvents or volatile monomers the pressure may also be higher.

Depending on the choice of polymerization conditions, the copolymers obtainable generally have weight-average molecular weights (M_{w}) in the range from 1000 to 200 000. In view of the use of the polymers, preference is given to those having a weight-average molecular weight of 5000 to 100 000. The weight-average molecular weight M_{w} can be determined in conventional manner by means of gel permeation chromatography, as elucidated in the examples. The K values of the copolymers obtainable in accordance with the invention, as determined by the method indicated below, are preferably in the range from 20 to 45.

Where the process is carried out as a solution polymerization in water, for many applications the removal of the water is unnecessary. Otherwise, the polymer obtainable in accordance with the invention can be isolated in conventional manner, as for example by spray drying of the polymerization mixture. Where the polymerization is carried out in a steam-volatile solvent or solvent mixture, the solvent can be removed by introducing steam, to give an aqueous solution or dispersion of the copolymer.

The resulting polymers and copolymers have a uniform molar weight distribution. The weight-average molar weight Mw and the number-average molar weight Mn are determined by means of gel permeation chromatography.

### The Use

The copolymers are preferably obtained in the form of an aqueous dispersion or solution. The solids content is preferably 10% to 80%, in particular 30% to 65% by weight.

The copolymers, particularly the copolymers of (meth)acrylic acid with (poly-C₂-C₄ alkylene glycol)-mono(meth)acrylic acid, preferably the copolymers of methacrylic acid with polyethylene glycol mono(C₁-C₁₀ alkyl) monomethacrylates, are outstandingly suitable as admixtures for cementitious preparations, such as concrete or mortar, and are notable in particular for superior properties in respect of their plasticizing action. The present invention accordingly further provides the copolymers obtainable by the process of the invention, and particularly copolymers of polyethylene glycol mono(C₁-C₁₀ alkyl) monomethacrylate with methacrylic acid, and also provides for their use in cementitious preparations, especially as concrete plasticizers.

By cement is meant for example Portland cement, high-alumina cement or mixed cement, such as, for example, pozzolanic cement, slag cement or other types. The copolymers of the invention are suitable in particular for cement mixes which as cement constituents comprise Portland cement predominantly and in particular at 80% by weight at least, based on the cement constituent. For this purpose the copolymers of the invention are used generally in an amount of 0.01% to 10% by weight, preferably 0.05% to 3% by weight, based on the total weight of the cement in the cement preparation.

The copolymers can be added in solid form or as an aqueous solution to the ready-to-use cementitious preparation. It is also possible to formulate copolymers that are present in solid form with the cement and to use such formulations to prepare the ready-to-use cementitious preparations. The copolymer is used preferably in liquid form, i.e., in dissolved, emulsified or suspended form, in the form for example of the polymerization solution, when preparing the preparation, i.e., during mixing.

The copolymers can also be used in combination with the known concrete plasticizers and/or concrete superplasticizers based on naphthalene/formaldehyde condensate sulfonate, melamine/formaldehyde condensate sulfonate, phenolsulfonic acid/formaldehyde condensate, lignosulfonates, and gluconates. Additionally they can be used together with celluloses, alkylcelluloses or hydroxyalkylcelluloses for example, or with starches or starch derivatives. They can also be employed in combination with high molecular weight polyethylene oxides (weight-average molecular weight M_{w} in the range from 100 000 to 8 000 000).

The cementitious preparation may further be admixed with typical additives such as air entrainers, expansion agents, water repellents, setting retardants, setting accelerants, antifreeze agents, waterproofing agents, pigments, corrosion inhibitors, plasticizers, grouting aids, stabilizers or hollow microspheres. Such additives are described for example in EN 934.

In principle the copolymers can also be used together with film-forming polymers. By these are meant polymers whose glass transition temperature is ≦65° C., preferably ≦50° C., more preferably ≦25° C., and very preferably ≦0° C. On the basis of Fox's (T. G. Fox, Bull. Am. Phys. Soc. (Ser. II) 1, 1956, 123) postulated relationship between the glass transition temperature of homopolymers and the glass transition temperature of copolymers, a person skilled in the art is able to select appropriate polymers. Examples of appropriate polymers are the styrene acrylates and styrene-butadiene polymers that are available commercially for this purpose (see, for example, H. Lutz in D. Distler (editor), “Wässrige Polymerdispersionen” Wiley-VCH, Weinheim 1999, sections 10.3 and 10.4, pp. 230-252).

Furthermore, it is often advantageous if the copolymers are used together with antifoams. Such use prevents excessive air in the form of air voids being introduced into the concrete during the preparation of the ready-to-use mineral building materials, since such air would lower the strength of the set mineral building material. Suitable antifoams comprise, in particular, polyalkylene oxide-based antifoams, trialkyl phosphates, such as tributyl phosphate, and silicone-based defoamers. Likewise suitable are the ethoxylation products and the propoxylation products of alcohols having 10 to 20 carbon atoms. Likewise suitable are the diesters of alkylene glycols and/or polyalkylene glycols, and also further typical antifoams. Antifoams are used typically in amounts of 0.05% to 10% and preferably of 0.5% to 5% by weight, based on the polymers.

The antifoams can be combined with the polymer in a variety of ways. If, for example, the polymer is in the form of an aqueous solution, the antifoam can be added in solid or dissolved form to the polymer solution. If the antifoam is not soluble in the aqueous polymer solution, then emulsifiers or protective colloids can be added in order to stabilize it.

If the copolymer is in the form of a solid, as obtained, for example, from a spray-drying or fluidized-bed spray-granulating operation, then the antifoam can be mixed in as a solid or else compounded together with the polymer in the course of the spray-drying or spray-granulating operation.

The examples which follow are intended to illustrate the invention.

### Analysis:

### a) Determination of K Value:

The K values of the aqueous sodium salt solutions of the copolymers were determined according to H. Fikentscher, Cellulose-Chemie, volume 13, 58-64 and 71-74 (1932) in aqueous solution at a pH of 7, a temperature of 25° C., and a polymer concentration of the sodium salt of the copolymer of 1% by weight.

### b) Determination of Solids Content:

The determination takes place by means of the Sartorius MA30 analytical instrument. A defined amount of the sample (approximately 0.5 to 1 g) is weighed out for this purpose into an aluminum boat and dried at 90° C. to constant weight. The percentage solids content (SC) is calculated as follows: SC=final mass×100/initial mass [% by weight]

### c) Determination of Molecular Weight:

The number-average and weight-average molecular weights were determined by means of gel permeation chromatography (GPC) using aqueous eluents.
The GPC was carried out using a system of apparatus from Agilent (1100 series). This system comprises:

| | |
|---|---|
| gasifier | model G 1322 A |
| isocratic pump | model G 1310 A |
| autosampler | model G 1313 A |
| column oven | model G 1316 A |
| control module | model G 1323 B |
| differential refractometer | model G 1362 A |

The eluent used in the case of polymers in solution in water is a 0.08 mol/l TRIS buffer (pH=7.0) in distilled water+0.15 mol/l chloride ions from NaCl and HCl. Separation took place in a separating column combination. The columns used are columns 789 and 790 (each 8×30 mm) from TosoHAAS, with GMPWXL separation material. The flow rate was 0.8 ml/min at a column temperature of 23° C.
Calibration is carried out using polyethylene oxide standards from the company PPS, with molecular weights M of 194-1 700 000 [ mol/g].

### d) NMR Analysis (Determining the Conversion)

For determining the conversion of the polyalkylene glycol, samples of the reaction mixture were taken at different times, and were admixed with a little trifluoroacetic acid. The samples were analyzed by means of ¹H NMR spectroscopy at 20° C., the reference signal used being the signal of the end group of the polyalkylene glycol (in the case of a polyalkylene glycol methyl ether, the signal at 3.4 ppm), which is coincident for the reactant and for the product. For determination of conversion, the integral of a signal which is characteristic of the reaction product, generally the signal of the methylene protons on the oxygen of the ester group (in general at about 4.3 ppm), was determined and was placed in relation to the integral of the end group.

### PREPARATION EXAMPLES

### Comparative Example

A 1 l glass reactor with anchor stirrer, thermometer, gas introduction line, reflux condenser, and dropping funnel was charged with 450 g of methyl polyethylene glycol (M=5000 g/mol), 90 mg of 2,6-di-tert-butyl-4-methylphenol, 9 mg of 4-hydroxy-N,N-2,2,6,6-tetramethylpiperidine-1-oxyl, and 1.59 g of sodium carbonate (anhydrous). The mixture was heated to 90° C. with introduction of air. Then 17.36 g of methacrylic anhydride were added and the reaction mixture was allowed to react at 90° C. for 2 hours. Subsequently the conversion was examined by means of ¹H NMR spectroscopy (100%) and the batch was diluted with 256 g of water and cooled to room temperature. Polymerization was carried out immediately after esterification.

Polymerization:

A 1 l glass reactor with anchor stirrer, thermometer, nitrogen introduction line, reflux condenser, and a plurality of feed vessels was charged with 290 g of water and this initial charge was heated to 60° C. Then, while introducing nitrogen and stirring, at an internal temperature of 60° C., feed stream 1 was added continuously over the course of 4 h and feed stream 2 over the course of 4.5 h, beginning simultaneously. After the end of the feeds, the copolymerization was completed by allowing the contents of the reactor to continue polymerization for 1 hour, after which they were cooled and neutralized with 25% strength aqueous sodium hydroxide solution.

Feed stream 1: Mixture of 250 g of the ester solution with 4.57 g of methacrylic acid and 0.41 g of mercaptoethanol.

Feed stream 2: 1.08 g of aqueous sodium peroxodisulfate solution (7% by weight), 14 mg of water

The solution obtained had a solids content of 29.6% by weight and a pH of 6.6. The K value of the polymer was 94.8, the number-average molecular weight Mn was 19 700, and the weight-average molecular weight Mw was 760 000 daltons (ratio Mw/Mn, as a measure of the uniformity: 38.6)

### Inventive Example

A 1 l glass reactor with anchor stirrer, thermometer, gas introduction line, reflux condenser, and dropping funnel was charged with 565 g of methyl polyethylene glycol (M=5000 g/mol), 110 mg of 2,6-di-tert-butyl-4-methylphenol, 11 mg of 4-hydroxy-N,N-2,2,6,6-tetramethylpiperidine-1-oxyl, and 1.99 g of sodium carbonate (anhydrous). The mixture was heated to 90° C. with introduction of air. Then 17.36 g of methacrylic anhydride were added and the reaction mixture was allowed to react at 90° C. for 2 hours. Subsequently the conversion was examined by means of ¹H NMR spectroscopy (100%) and the batch was diluted with 256 g of water with 2.26 g of hypophosphorous acid as reducing agent, and cooled to room temperature. Polymerization was carried out immediately after esterification.

Polymerization:

A 1 l glass reactor with anchor stirrer, thermometer, nitrogen introduction line, reflux condenser, and a plurality of feed vessels was charged with 280 g of water and this initial charge was heated to 60° C. Then, while introducing nitrogen and stirring, at an internal temperature of 60° C., feed stream 1 was added continuously over the course of 4 h and feed stream 2 over the course of 4.5 h, beginning simultaneously. After the end of the feeds, the copolymerization was completed by allowing the contents of the reactor to continue polymerization for 1 hour, after which they were cooled and neutralized with 25% strength aqueous sodium hydroxide solution.

Feed stream 1: Mixture of 241 g of the ester solution with 4.44 g of methacrylic acid and 0.49 g of mercaptoethanol.

Feed stream 2: 1.05 g of aqueous sodium peroxodisulfate solution (7% by weight), 14 mg of water

The solution obtained had a solids content of 29.4% by weight and a pH of 6.7. The K value of the polymer was 52.4, the number-average molecular weight Mn was 17 300, and the weight-average molecular weight Mw was 164 000 daltons (ratio Mw/Mn, as a measure of the uniformity: 9.5)

1. A process for preparing a free-radically polymerizable carboxylic ester, comprising reacting a carboxylic acid component comprising an ethylenically unsaturated carboxylic acid, a carboxylic anhydride, or a carbonyl halide with a hydroxyl compound comprising a polyalkoxy compound comprising at least 60% by weight of C₂ to C₄ alkoxy groups wherein said reacting is carried out
in the presence of a polymerization inhibitor and a reducing agent.

2. The process according to claim 1, wherein the carboxylic acid component is maleic acid, itaconic acid, fumaric acid, acrylic acid, methacrylic acid or anhydrides thereof.

3. The process according to claim 1, wherein the polyalkoxy compound comprises at least 80% by weight of ethoxy groups, propoxy groups or mixtures thereof and has one or two hydroxyl groups.

4. The process according to claim 1, wherein the polyalkoxy compound is polyethylene glycol mono(C₁-C₁₀ alkyl)ethers having a number-average molar weight of 400 to 10 000.

5. The process according to claim 1, wherein the polymerization inhibitor comprises oxygen to form free radicals.

6. The process according to claim 1, wherein the reducing agent is a phosphorus compound, sulfur compound, hypophosphorous acid or salts thereof.

7. The process according to claim 1, comprising carrying out said reacting in the presence of a gas mixture having an oxygen concentration of 1% to 15% by volume.

8. The process according to claim 1, comprising carrying out said reacting in the presence of a base.

9. The process according to claim 1, wherein the reaction mixture comprises less than 5% by weight of water.

10. The process according to claim 1, comprising carrying out said reacting in the presence of less than 5% by weight of water and/or organic solvents.

11. A process for preparing a homopolymer or copolymer, comprising free radically polymerizing one or more carboxylic esters according to claim 1 as monomers.

12. A process for preparing a homopolymer or copolymer, comprising free radically polymerizing one or more carboxylic esters according to claim 1 without prior isolation from an esterification product mixture, wherein a monomer is added to said product mixture.

13. The process according to claim 11, comprising synthesizing the copolymer from:
10% to 99.9% by weight of the free-radically polymerizable carboxylic ester according to claims 1,
0.1% to 50% by weight of acrylic acid or methacrylic acid, and
0% to 30% by weight of further monomers.

14. A copolymer obtained by a process according to claim 11.

15. A cement comprising a copolymer according to claim 14 as a plasticizing additive.
